SEQUENCE LISTING (5 sequences)

SEQ ID NO: 1 - DNA, 918 bp, synthetic construct
  source 1..918: mol_type = other DNA
atgggcagactgacaagcggcgtgggcacagcagctctgctggtcgtggccgttggcctgagagtggtgtgcgccaagtatgccctggccgacccctccctgaaaatggccgatcctaacagattccggggaaagaatctgcctgtgctggaccagctgacagatccacccggcgtgaaaagagtgtaccacatccagccttctctggaagatcctttccagcctcccagcatcccaatcaccgtgtactacgccgtgctggaacgcgcttgccggtctgtgctgctgcacgcacctagcgaggcccctcagatcgtgcggggcgccagcgacgaggctagaaagcacacctacaacctcacaatcgcctggtaccggatgggcgacaattgcgctatccctatcaccgtgatggaatacaccgagtgcccctataacaagagcctgggcgtgtgtcctattcggacccaacccagatggtcctactacgacagcttttctgccgtgagcgaagataacctgggattcctgatgcacgcccccgccttcgagacagccggaacctacctgagactggtgaagatcaacgactggaccgagatcacccaatttatcctggagcatagagccagagctagctgcaagtacgccctgcctctgagaatccctcccgccgcctgtctgaccagcaaggcctaccagcagggcgtcacagtggacagcatcggcatgctgcctaggttcatccccgaaaaccagagaaccgtggccctgtacagcctgaagatcgccggctggcacggccccaagcctccatacaccagcaccctgctgccacctgaactttctgataccaccaacgccacacagcctgagctggtgcctgaggaccctgaggactccgccctgctggagtga

SEQ ID NO: 2 - DNA, 1182 bp, synthetic construct
  source 1..1182: mol_type = other DNA
atgggaagacttacatccggcgtgggcaccgccgctttgctggttgtggccgtgggcctgagagtggtgtgcgccaagtacgccctggccgatccttctctgaagatggccgaccccaatagattccgaggcaagaacctgcctgtgctggaccagctgaccgatccccctggcgtgaagcgggtgtaccacattcagccttccctggaggacccattccagcctcctagcatcccaatcacagtctactatgccgtgctggaaagagcttgtagaagcgtgctgctgcatgccccttctgaagccccccagatcgtgagaggagcctctgacgaggcccggaagcacacatacaatctgacaatcgcctggtacagaatgggcgacaactgcgccattcctatcaccgtcatggaatacaccgagtgcccctacaacaagagcctgggagtgtgtcctatccggacccagcccagatggagctactacgacagcttcagcgccgtgtccgaggacaacctgggcttcctgatgcacgccccagcttttgagacagccggcacctacctgcggctggtgaaaatcaacgactggaccgagatcacccaatttatcctggaacacagagccagagccagctgcaagtacgccctgccactgagaatcccccccgctgcctgcctgaccagcaaggcctaccagcagggcgtgaccgtggacagcatcggcatgctgcctcggttcatccccgagaaccagagaacagtggctctgtacagcctcaagatcgctggctggcacggccctaaacctccttatacaagcaccctgctgcctcctgaactgagcgacaccaccaacgccacacaacctgagctggtgcctgaggatcccgaggatagcgccctgctggaagatcccgctggcacagtttctagccagatcccacctaactggcacatcccctccatccaggacgtggcccctcaccacgctcctgccgcccctagcaaccccggactgatcatcggcgccctggctggttctaccctcgccgtgctggtgatcggcggcatcgccttctgggtgcggagaagggcccagatggcccctaaaagactgcggctgccccacatcagagacgatgacgctcctcctagccaccagcctctgttctactga

SEQ ID NO: 3 - DNA, 3825 bp, synthetic construct
  source 1..3825: mol_type = other DNA
atgttcgtgttcctggtcctgttgcccctggtgtctagccagtgcgtgaatctgcggaccagaacccagctgccccccgcttataccaactccttcaccagaggagtgtactaccccgataaggtgttccggtcctctgtgctgcacagcacccaggacctgttcctgcctttcttcagcaacgtgacctggttccacgccatccacgtgtccggcacaaacggcacgaaaagattcgacaaccctgtgctaccttttaacgatggcgtatatttcgcctccaccgagaaatctaacatcatcagaggctggatattcggcaccaccctggacagcaagacccagagtctgctgattgtgaacaacgccaccaatgtggtgattaaggtgtgcgagtttcagttttgtaacgaccctttcctggacgtgtactaccacaagaataacaaaagctggatggaaagcggcgtgtactctagcgctaacaactgcacattcgagtacgtgagccagcctttcctgatggacctggaaggcaaacagggaaacttcaagaacctcagagaattcgtgtttaagaacatcgacggctacttcaagatctatagcaagcacacccccatcaacctggtgcgcgatctgccccagggcttttctgccctggaacccctggtggacctgcctatcggcattaacatcaccagattccaaaccctgctggccctgcatagaagctacctgacacctggagatagcagcagcggctggaccgccggcgctgccgcttactacgtgggatacctgcagccacgaacattcctgctgaagtacaacgagaacggcacaattaccgatgccgtggactgcgccctggatcctctctctgaaaccaaatgcaccctgaagtctttcacagttgagaagggcatctatcagaccagcaactttcgggtgcagcctaccgagagcatcgtgaggtttcctaatatcactaacctgtgtcctttcggcgaggtgtttaacgccacaagattcgcctccgtgtatgcttggaacagaaagcggattagcaactgcgtggctgactacagcgtcctgtacaacagcgctagcttctccaccttcaagtgctacggcgtgtccccaaccaagctgaacgacctgtgcttcactaacgtgtacgccgacagcttcgtgatcagaggcgacgaagtccgacagatcgcaccaggacaaacagggaagatcgccgattacaactacaagctgccagacgacttcaccggttgtgtgatcgcctggaacagtaataatctggacagcaaggtgggaggcaactacaactacagatacagactgtttagaaagagcaatctgaaacctttcgagcgggacataagcaccgagatttaccaggccggctcaaaaccttgcaacggcgtggaaggcttcaactgctacttccctctccaatcttacggcttccagccaacaaacggcgttggctaccaaccttaccgggttgtcgtgctgagcttcgagctgctgcacgcccctgccacagtctgcggccctaagaagagcaccaacctcgtgaaaaacaaatgcgtgaatttcaacttcaacggcctgaccggcaccggtgtgctcacagaaagtaacaaaaagttcctgccttttcagcagttcggcagagatatcgctgacaccaccgatgccgtccgggacccccagacactggaaatcctggatatcaccccttgtagcttcggcggagtgagtgtgataacccctggcaccaacaccagcaaccaggtggctgtgctgtaccagggcgttaattgtaccgaagtgcccgtggccatccacgccgatcagctgacacctacatggcgggtgtactccaccggctctaatgttttccaaacacgggccggctgtctgatcggagccgagcacgtgaacaacagctacgagtgtgacatccccatcggcgccgggatctgcgccagctaccagacccaaacgaactcccctggatctgcctcatcggtggcctcccagagcatcatcgcctacaccatgtcgctgggagccgaaaacagcgtggcctactccaacaacagcatcgccatccctaccaacttcaccatcagcgtgaccacagagatcctgcccgtgagcatgaccaagacaagcgtggactgcaccatgtacatctgcggcgacagcaccgagtgcagcaatctgctgctgcagtatggcagcttctgtacacagctgaacagagcccttacaggcatagcagttgagcaggacaagaatacacaagaggtgtttgcccaagtgaagcagatctacaagaccccacctatcaaggacttcggaggattcaatttcagccagatcctgcccgaccccagcaagcctagcaagcggtccttcatcgaagatctgctgttcaacaaggtgaccctggctgacgccggctttatcaagcagtacggcgattgcctgggcgacatcgccgctagagatttgatttgtgcccagaagttcaacggcctgaccgtgctgcctcctctgctgaccgacgagatgatcgcccagtacaccagcgctctgcttgccggaaccatcacaagcggctggacattcggcgccggcgccgcactgcagatcccctttgccatgcagatggcctacagattcaatggcatcggggttacccagaacgtgctctatgaaaatcagaagctgatcgccaaccagttcaactctgccatcggcaaaatccaggatagcctgagcagcacagcttctgccctgggcaagctgcagaacgtggtcaatcagaatgcccaggccctgaacacactggtgaagcagttatcttcaaatttcggcgccatctcttctgttctgaacgacatcctgtcaagactggaccctccagaggccgaggtgcagatcgatagactgatcacaggccggctgcaaagcctccaaacctacgtgacacagcaacttatcagggctgccgagatcagagccagcgccaacctggccgccaccaagatgagcgagtgcgtgctgggccagagcaagagagtggacttttgcggcaaaggctaccacctgatgagcttccctcagtctgcccctcacggcgtggtctttctgcacgtgacatacgtgcctgctcaggagaagaacttcacaaccgcccctgccatctgccacgatggcaaggcccatttcccccgcgaaggcgtattcgtgtccaacggaacccactggttcgtgacacagcggaacttctacgagcctcagattatcaccaccgacaataccttcgtgagcggcaactgcgacgtggtcatcggcatcgtgaacaataccgtgtacgaccccctgcagcccgagctggacagctttaaggaagagctggataaatacttcaagaaccacacaagccctgacgtcgacctgggcgatatcagcggcatcaacgcctctgtcgtgaacatccagaaggagatcgacagactgaatgaagtggctaagaacctcaacgagagcctgatcgacctgcaagagctgggaatgaacacccagtttgaggccgtgggccgggagtttaacaacctggaacgtaggatcgagaacctgaacaagaagatggaggactgctgcctggatgtgtggacttacaatgccgagctgttggtgctgatggaaaacgagagaacactggatttccacgactctaatgtgaaaaacctgtacgacaaggtgcgactccagctgagagataatgcctga

SEQ ID NO: 4 - DNA, 37 bp, synthetic construct
  source 1..37: mol_type = other DNA
agggaataagggcgacacggaaatgttgaatactcat

SEQ ID NO: 5 - DNA, 32 bp, synthetic construct
  source 1..32: mol_type = other DNA
ggattgggaagacaatagcaggcatgctgggg

(19) United States
(12) Patent Application Publication
(10) Pub. No.: US 2024/0091343 A1
(43) Pub. Date: Mar. 21, 2024

(54) TECHNOLOGY PLATFORM OF UNCAPPED-LINEAR MRNA WITH UNMODIFIED URIDINE

(71) Applicant: Binhui Biopharmaceutical Co., Ltd, Wuhan (CN)

(72) Inventors: Binlei LIU, Wuhan (CN); Linkang CAI, Wuhan (CN); Peng NI, Wuhan (CN)

(21) Appl. No.: 18/455,035

(22) Filed: Aug. 24, 2023

(30) Foreign Application Priority Data
Jul. 18, 2022 (CN) 202210839969.2

(51) Int. Cl. A61K 39/215; A61K 9/127; A61K 39/245; A61P 37/04; C12N 7/00; C12N 15/85

(52) U.S. Cl. CPC A61K 39/215; A61K 9/1272; A61K 39/245; A61P 37/04; C12N 7/00; C12N 15/85; A61K 2039/53

(57) ABSTRACT

Provided are a method for preparing an mRNA-LNP and use thereof, on which a technology platform of an uncapped-linear mRNA with unmodified uridine is based. The provided linear mRNA is composed of a 5′ UTR including the IRES, a coding region and a poly A region has a simple structure and high stability and efficiency on its expression in vivo and in vitro. The uncapped-linear mRNA with unmodified uridine is then encapluslated by a new lipid nanoparticle (LNP-1) to form an mRNA-LNP.

### CROSS-REFERENCE TO RELATED APPLICATIONS

This application claims priority to and the benefit of Chinese Patent Application No. 202210839969.2 filed on Jul. 18, 2022, the entire contents of which are incorporated herein by reference. This application is also related to Chinese Patent Application No. 202210521239.8 filed on May 13, 2022, the entire contents of which are incorporated herein by reference.

### SEQUENCE LISTING

This application contains a Sequence Listing XML submitted under the provisions of 37 CFR 1.831(a). The Sequence Listing XML entitled “BIOC2307236US-SEQUENCE LISTING.xml” was prepared on Aug. 17, 2023 and is 12 KB in size. The above-referenced Sequence Listing XML is incorporated herein by reference in its entirety.

### FIELD

The present disclosure relates to the technical field of mRNA preparation, and specifically relates to a construction method of a technology platform of an uncapped-linear mRNA with unmodified uridine and use thereof, where the use includes preparing a vaccine or medicament by the technology platform of an uncapped-linear mRNA with unmodified uridine.

### BACKGROUND

Messenger RNA (mRNA) is responsible for transcribing the genetic information stored in DNA and directing the synthesis of proteins in cells. In theory, once transcribed and delivered into the cytoplasm, mRNA with modifications can be translated to generate any protein desired, by using amino acids in the cytoplasm. However, mRNA has various problems, such as extreme instability, the propensity to trigger unnecessary immune responses, degradation by RNases that occurs widely in vivo and in vitro, and dependency on the translation mechanism in the target cells. Efforts have been made for decades to overcome these shortcomings of mRNA.

Studies have found that many naturally existing mRNA chemical modifications have biological significance in mRNA stability. At present, the following chemical modifications are commonly used in mRNA technology: N1- and N6-methyladenosine (m1A, m6A, m6Am), 3- and 5-methylcytosine (m3C, m5C), 5-hydroxymethylcytosine (hm5C), 2′-O-methylation (Nm) and pseudouridine (Ψ).

Among these chemical modifications, the methyl group on m1A blocks Watson-Crick base pairing, thereby changing the mRNA structure and promoting translation initiation; m6A is a reversible dynamic RNA modification and an indispensable part of RNA metabolism regulation, which can regulate stability, splicing, transport, localization, and translation of mRNA; m5C affects mRNA structural stability and translation efficiency and can promote mRNA nuclear export; Nm is a cotranscriptional or posttranscriptional modification of mRNA, which can increase the hydrophobicity of mRNA, protect it from nuclease attack, stabilize the helical structure of mRNA, and affect the interaction of mRNA with proteins or other RNAs; Ψ is the first discovered and most common mRNA modification. Katalin Karikó et al. have successively found that the introduction of pseudouridine triphosphate (ΨTP) into mRNA can reduce its immunogenicity, which decreases with the increase in the proportion of ΨTP introduced. An mRNA in which uridine triphosphate (UTP) is completely replaced with ψTP can not only greatly reduce the immunogenicity of mRNA but also improve the stability of mRNA and enhance its translation efficiency. In 2015, Oliwia Andries et al. found that the complete replacement of UTP with N1-methylpseudouridine triphosphate (NlmψTP) reduced the immunogenicity of mRNA and enhanced protein expression more than the complete replacement of UTP with ψTP. The two new coronavirus mRNA vaccines, mRNA-1273 (Moderna) and BNT162b2 (Pfizer-BioNTech), both use NlmψTP to replace UTP.

Eukaryotic mRNA structures generally have a 5′ cap, a 5′-untranslated region (UTR), a coding region, a 3′-UTR and a 3′ tail (i.e. polyadenosine tail, poly A or pA). The 5′ cap closes the 5′ end of mRNA thereby preventing degradation by exonucleases and may be a recognition site of the protein synthesis system to be recognized and bond by cap binding protein eIF-4E, so as to promote mRNA to combine with small subunits of ribosomes to initiate translation, playing an important role of enhancing stability and promoting translation of the mRNA. Generally, cap structural analogs may be used for capping after transcription, for example, using m7GpppG for cotranscriptional capping or using capping enzymes such as vaccinia capping enzyme (VCE) alone for capping after transcription. In addition to 5′ cap for mediating the combination with ribosomes and translation improvement, the internal ribosome entry site (IRES) has a similar function to the cap structure. In 1988, Pelletier et al. found that the 5′-UTR of poliovirus has a P2 sequence of approximately 450 nucleotides that can guide eukaryotic mRNA translation; Jang et al. also found a sequence in the 5′-UTR of encephalomyocarditis virus that can guide internal entry of ribosome to initiate translation. In 1991, Macejak et al. found that there is an IRES element in the 5′-UTR of the cellular immunoglobulin heavy chain binding protein gene. It was demonstrated that IRES can guide protein synthesis in a circular mRNA, indicating that protein translation can specifically depend on the IRES sequence. IRES facilitates ribosome assembly and initiates translation by recruiting different trans-acting factors, making it possible for protein translation initiation to occur independently of the 5′ cap structure of the mRNA.

Complementary to a working mRNA technology is a proper delivery technology. Since the mRNA chain is a negatively charged long-chain macromolecule and the surface of the cell membrane is also negatively charged, electrostatic repulsion makes it difficult for the mRNA molecule to pass through the cell membrane and enter the cell. In addition, mRNA, as a single-chain molecule, is very unstable and may be quickly degraded by a variety of enzymes in the body. Therefore, the delivery of mRNA to the interior of cells requires addressing enzymatic degradation and membrane barriers caused by electrostatic repulsion.

With the approval of two mRNA vaccines, lipid nanoparticles (LNPs) are acknowledged as the most successful mRNA delivery vehicles. Liposomes have vesicle structures composed of lipid molecules that were discovered by scientists A. D. Bangham and R. W. Horne under a microscope as early as 1961. In 1995, the first liposomal drug Doxil approved by the FDA in the U.S. was used for ovarian cancer and breast cancer chemotherapy through the drug doxorubicin encapsulated by HSPC/DMG-PEG dual component liposome to reduce the toxicity of free drugs to other organs. In 2018, the FDA in the U.S. approved Onpattro, a drug that encapsulates nucleic acid fragments (siRNA) with LNPs, for the treatment of hereditary transthyretin-mediated amyloidosis of polyneuropathy. For the mRNA vaccine against the new COVID-19 coronavirus, Moderna used an ionizable lipid SM-102 developed independently, while Pfizer and BioNTech used an ionizable lipid named ALC-0315. LNPs can not only effectively deliver mRNA to specific target cells to prevent mRNA from being degraded or cleared but also help mRNA escape from cellular endosomes to the cytoplasm in a timely manner to be translated into corresponding proteins.

The application of mRNA as a new generation of vaccine drugs will be more extensive. However, in the existing technology and application of preparing vaccine with mRNAs, not all of modified mRNAs show strong ability for protein expression. In addition, there is also a lack of efficient and stable delivery vector to deliver mRNA to specific cells to promote its translation into target protein.

In view of this, it is of high requirement to construct an efficient and high stable technology platform, to provides a method and application foundation for the preparation of mRNA, thereby promoting the further development of mRNA technology.

### SUMMARY

For the technical problems above, the present disclosure aims at providing an mRNA technology platform which provides technical support on the preparation of mRNA and use of the prepared mRNA as a vaccine.

According to the technical concept above, the present disclosure seeks solutions in terms of each of steps including synthesis, modification and delivery in the preparation of mRNA vaccine, in which the modification and delivery of mRNA especially affects the expression of the mRNA at last, i.e. the pharmaceutical effect of mRNA as a vaccine drug.

Regarding to the modification of mRNA, the present disclosure provides a modification method with uncapped structure, where the IRES with similar function to the cap structure is used, rather than the conventional method for replacing UTP with ΨTP. The IRES can mediate the combination with ribosomes and promote translation of mRNA, presenting higher expression efficiency than that of the capping in the conventional method. For technical implementation, the IRES sequence is introduced into a plasmid, one end of the IRES sequence is connected with a promoter sequence, and the other end of the IRES sequence is connected with a gene of interest (GOD to construct a recombinant plasmid, which is used for amplifying the DNA template encoding desired mRNAs, or is taken, after linearization, as the DNA template for amplifying the desired mRNAs, and thus producing linearly desired mRNAs by transcription of the DNA template finally.

The conventionally prepared mRNA includes a 3′UTR between the coding region and poly A region, and previous research demonstrated that the 3′UTR enhances stability of mRNA. In embodiments of the present disclosure, the 3′UTR is removed from the prepared mRNA, but the prepared mRNA still presents high expression in the translation. Thus, the mRNA provided in embodiments of the present disclosure has a simple structure and high efficiency on its preparation and expression.

Regarding to the delivery of mRNA, the present disclosure provides a new LNP as a delivery vehicle for mRNA, in which the LNP as the delivery vehicle can specifically and effectively deliver the mRNA into target cells to prevent the mRNA from being degraded or cleared. Furthermore, the LNP can help mRNA escape from cellular endosomes to the cytoplasm in a timely manner to be translated into corresponding proteins.

According to the technical concept above, embodiments of the present disclosure provide a method for preparing an mRNA-LNP and use thereof, where the mRNA-LNP refers to a LNP encapsulating an mRNA. Based on such a method and the use, the inventors of the present disclosure create a technology platform of an uncapped-linear mRNA with unmodified uridine which is efficient and high stable.

In a first aspect, embodiments of the present disclosure provide uncapped mRNA with a linear structure sequentially formed by regions A, B and C, each of the regions A, B and C including one or more of an adenine ribonucleotide, a guanine ribonucleotide, a cytosine ribonucleotide and a uracil ribonucleotide, where the region A is a 5′-untranslated region (UTR) comprising an internal ribosome entry site (IRES) for mediating internal entry of a ribosomal subunit so as to guide translation of the mRNA; the region B is a coding region for the translation so as to generate a protein; and the region C is a poly A region for mediating a translation efficiency of the mRNA and enhancing stability of the mRNA.

In a second aspect, embodiments of the present disclosure provide a recombinant plasmid for preparing an uncapped mRNA in the embodiments of the first aspect, wherein the recombinant plasmid is any one of the following recombinant plasmids A, B and C: the recombinant plasmid A containing: a truncated D-type envelope glycoprotein ectodomain coding gene (gD^{ED}) from herpes simplex virus type 2 (HSV2), as set forth in SEQ ID NO: 1, or a sequence having at least 90% identity with SEQ ID NO: 1 and encoding a protein having the same activity as that of the gD^{ED} protein; the recombinant plasmid B containing: a truncated D-type envelope glycoprotein coding gene (gD^{FR}) from HSV2, as set forth in SEQ ID NO: 2, or a sequence having at least 90% identity with SEQ ID NO: 2 and encoding a protein having the same activity as that of the gD^{FR} protein; and the recombinant plasmid C containing a mutated gene (S^{δT}) encoding Delta strain SARS-CoV-2 spike protein as set forth in SEQ ID NO: 3, or a sequence having at least 90% identity with SEQ ID NO: 3 and encoding a protein having the same activity as that of the S^{δT} protein.

In a third aspect, embodiments of the present disclosure provide an mRNA-LNP including a LNP and an mRNA encapsulated therein, where the mRNA is an uncapped mRNA described in the embodiments of the first aspect.

In embodiments of the present disclosure, the LNP is one or more of octanoic acid, 8[(2-hydroxyethyl)[6-oxo-6-(undecyloxy)hexyl]amino]-, 1-octylnonyl ester and lipid-1, wherein the lipid-1 has a structure as shown in formula I:

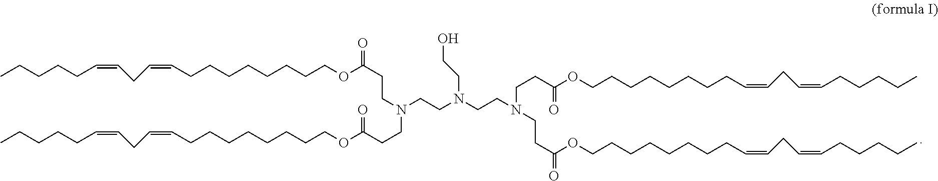 [SMILES: CCCCC/C=C\C/C=C\CCCCCCCCOC(=O)CCN(CCC(=O)OCCCCCCCC/C=C\C/C=C\CCCCC)CCN(CCO)CCN(CCC(=O)OCCCCCCCC/C=C\C/C=C\CCCCC)CCC(=O)OCCCCCCCC/C=C\C/C=C\CCCCC]

In a fourth aspect, embodiments of the present disclosure provide a method for preparing an mRNA-LNP, including: dissolving a cationic lipid compound and a helper molecule in ethanol at a molar ratio so as to obtain an ethanol phase; mixing the uncapped mRNA of the first aspect dissolved in a sodium acetate buffer with the ethanol phase at a volume ratio so as to obtain an mRNA-LNP mixture; and performing concentration and filtration sterilization on the mRNA-LNP mixture to obtain the mRNA-LNP.

In embodiments of the present disclosure, the cationic lipid compound is one or more of octanoic acid, 8[(2-hydroxyethyl)[6-oxo-6-(undecyloxy)hexyl]amino]-, 1-octylnonyl ester and lipid-1, wherein the lipid-1 has a structure as shown in formula I:

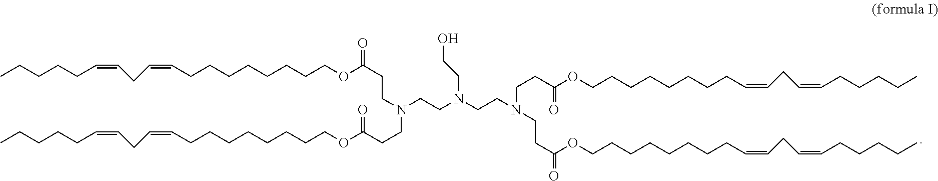 [SMILES: CCCCC/C=C\C/C=C\CCCCCCCCOC(=O)CCN(CCC(=O)OCCCCCCCC/C=C\C/C=C\CCCCC)CCN(CCO)CCN(CCC(=O)OCCCCCCCC/C=C\C/C=C\CCCCC)CCC(=O)OCCCCCCCC/C=C\C/C=C\CCCCC]

In embodiments of the present disclosure, the helper molecule is one or more of distearoyl phosphatidylcholine (DSPC), cholesterol and 1,2-dimyristoyl-rac-glycero-3-methoxypolyethylene glycol-2000, preferably, the helper molecule is distearoyl phosphatidylcholine, cholesterol and 1,2-dimyristoyl-rac-glycero-3-methoxypolyethylene glycol-2000.

In embodiments of the present disclosure, the helper molecule is DSPC, cholesterol and 1,2-dimyristoyl-rac-glycero-3-methoxypolyethylene glycol-2000 (DMG-PEG2000).

In embodiments of the present disclosure, the molar ratio of the cationic lipid compound to the helper molecule in the ethanol phase is of the cationic lipid compound, DSPC, cholesterol and DMG-PEG2000 at (40-55):(5-15):(30-45):(1-5), preferably at 50:10:38:2.

In embodiments of the present disclosure, the sodium acetate buffer the mRNA dissolved in has a concertation of 25 mM and a pH value for 5.

In embodiments of the present disclosure, the volume ratio of the sodium acetate buffer to the ethanol phase is 4:1.

In a fifth aspect, embodiments of the present disclosure provide use of an uncapped mRNA according to embodiments of the first aspect, a recombinant plasmid according to embodiments of the second aspect, an mRNA-LNP of according to embodiments of the third aspect, or an mRNA-LNP prepared according to a method according to embodiments of the fourth aspect in the manufacture of a vaccine or medicament.

In a sixth aspect, embodiments of the present disclosure provide a vaccine or medicament, containing an mRNA-LNP according to embodiments of the third aspect, or an mRNA-LNP prepared according to a method of according to embodiments of the fourth aspect and a pharmaceutically acceptable excipient.

In a seventh aspect, embodiments of the present disclosure provide a method for stimulating immune response in a subject, comprising administering an mRNA-LNP described in any one of embodiments in the third aspect to the subject, wherein the mRNA encapsulated by the LNP comprises an antigen coding gene selected from one or more of the follows: a truncated D-type envelope glycoprotein ectodomain coding gene (gD^{ED}) from herpes simplex virus type 2 (HSV2), as set forth in SEQ ID NO: 1; a truncated D-type envelope glycoprotein coding gene (gD^{FR}) from HSV2, as set forth in SEQ ID NO: 2; and a mutated gene (S^{δT}) encoding Delta strain SARS-CoV-2 spike protein as set forth in SEQ ID NO: 3.

In embodiments of the present disclosure, the LNP is one or more of octanoic acid, 8[(2-hydroxyethyl)[6-oxo-6-(undecyloxy)hexyl]amino]-, 1-octylnonyl ester and lipid-1, wherein the lipid-1 has a structure as shown in formula I:

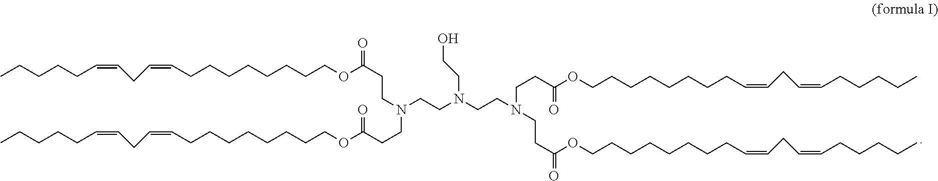 [SMILES: CCCCC/C=C\C/C=C\CCCCCCCCOC(=O)CCN(CCC(=O)OCCCCCCCC/C=C\C/C=C\CCCCC)CCN(CCO)CCN(CCC(=O)OCCCCCCCC/C=C\C/C=C\CCCCC)CCC(=O)OCCCCCCCC/C=C\C/C=C\CCCCC]

In embodiments of the present disclosure, the subject is human or non-human such as mouse and rabbit.

In embodiments of the present disclosure, the immune response comprises activation of T cells specific to the antigen.

In embodiments of the present disclosure, the mRNA-LNP is administered intramuscularly, intracutaneously, subcutaneously, intravenously or intraperitoneally to the subject, preferably the mRNA-LNP is administered intramuscularly or intracutaneously.

Compared with the prior art, the present disclosure has at least the following beneficial effects.

The present disclosure provides in embodiments a method for preparing an mRNA-LNP and use thereof, Based on which the present disclosure creates an efficient and high stable mRNA technology platform. The provided mRNA with a linear structure composed of a 5′ UTR including the IRES, a coding region and a poly A region has a simple structure and high stability and efficiency on its expression in vivo and in vitro. For example, three recombinant plasmids were constructed with introductions of three GOIs, i.e., two truncated D-type envelope glycoprotein coding gene from herpes simplex virus type 2 (HSV2) and a mutated gene encoding Delta strain SARS-CoV-2 spike protein, respectively, followed by preparing three mRNAs based on the three recombinant plasmids, then to form mRNA-LNPs by encapsulating the three prepared mRNAs into new LNPs individually. The method for preparing the mRNA-LNP is of high efficiency and the prepared products is stable, of good performance and low preparation cost, and could be used for preparing vaccines or medicines.

### BRIEF DESCRIPTION OF THE DRAWINGS

FIG. 1 shows structures of recombinant plasmids according to embodiments of the present disclosure.

FIG. 2 is a plot of concentration of linear IVT template amplified by PCR according to embodiments of the present disclosure.

FIG. 3 shows an electrophoretic imaging of linear mRNAs synthesized based on the IVT template in agarose gel according to embodiments of the present disclosure.

FIG. 4 shows a comparison between the peaks of the linear mRNA before and after purification according to embodiments of the present disclosure.

FIG. 5 shows a process flow of mRNA-LNP preparation according to embodiments of the present disclosure.

FIG. 6 shows an electrophoretic imaging of the mRNA-LNP according to embodiments of the present disclosure.

FIG. 7 shows particle sizes with encapsulation according to embodiments of the present disclosure.

FIG. 8 shows fluorescence expression from eGFP-mRNA-LNP transfected into BHK cells in vitro according to embodiments of the present disclosure.

FIG. 9 shows fluorescence expression after in vitro transfecting eGFP-mRNA-LNP with SM102 or Lipid-1 according to embodiments of the present disclosure.

FIG. 10 shows the fluorescence intensity at several points in time based on Fluc-mRNA-LNP encapsulated by SM102 or Lipid-1 according to embodiments of the present disclosure.

FIG. 11 shows the fluorescence intensity at several points in time based on Fluc-mRNA-LNP encapsulated by SM102 according to embodiments of the present disclosure.

FIG. 12 is a line chart showing a total fluorescence of Fluc-mRNA-LNP encapsulated by Lipid-1 according to embodiments of the present disclosure.

FIG. 13 is a line chart showing a total fluorescence of Fluc-mRNA-LNP encapsulated by SM102 according to embodiments of the present disclosure.

FIG. 14 shows a comparison on fluorescence intensity after injection of Fluc-mRNA^{UTP}-LNP or Fluc-mRNA^{N1mψTP}-LNP in 4 hours according to embodiments of the present disclosure.

FIG. 15 is a histogram showing the fluorescence intensity after injection of Fluc-mRNA^{UTP}-LNP or Fluc-mRNA^{N1mψTP}-LNP in 4 hours according to embodiments of the present disclosure.

FIG. 16 shows time points for immunizations and blood collections for C57BL/6 mice according to embodiments of the present disclosure.

FIG. 17 shows serum IgG antibody titer of immunized C57BL/6 mice in an UTP group and NlmψTP group according to embodiments of the present disclosure.

FIG. 18 shows time points for immunizations and blood collections for Syrian hamsters according to embodiments of the present disclosure.

FIG. 19 shows serum IgG antibody titer of immunized Syrian hamsters in different UTP groups according to embodiments of the present disclosure.

FIG. 20 shows neutralizing antibody titer for Delta and Omicron strains of SARS-CoV-2 detected in the immunized Syrian hamsters in different UTP groups according to embodiments of the present disclosure.

FIG. 21 shows time points for immunizations with gD^{ED}-mRNA-LNP/gD^{FR}-mRNA-LNP and blood collections for C57BL/6 mice according to embodiments of the present disclosure.

FIG. 22 shows fluorescence expression by neutralizing oHSV2 virus in C57BL/6 mice immunized with gD^{ED}-mRNA-LNP/gD^{FR}-mRNA-LNP according to embodiments of the present disclosure.

FIG. 23 shows time points for immunizations and blood collections for Japanese white rabbits according to embodiments of the present disclosure.

FIG. 24 shows results of immunizations with gD-mRNA-LNP in the Japanese white rabbits according to embodiments of the present disclosure.

FIG. 25 is a histogram showing neutralization titers differential on the immunized dose and route in the Japanese white rabbits immunized with gD-mRNA-LNP according to embodiments of the present disclosure.

FIG. 26 shows time points for immunizations with gD-mRNA-LNP for C57BL/6 mice according to embodiments of the present disclosure.

FIG. 27 shows time points for immunizations with S^{δT}-mRNA-LNP for C57BL/6 mice according to embodiments of the present disclosure.

FIG. 28 shows spots formed by IFN-γ cytokines secreted by C57BL/6 mice immunized with gD^{ED}-mRNA-LNP or gD^{FR}-mRNA-LNP according to embodiments of the present disclosure.

FIG. 29 shows a comparison on the average number of spots formed by IFN-γ cytokines secreted by C57BL/6 mice immunized with gD^{ED}-mRNA-LNP or gD^{FR}-mRNA-LNP according to embodiments of the present disclosure.

FIG. 30 shows activations of T cells by S^{δT}-mRNA-LNP according to embodiments of the present disclosure.

FIG. 31 shows the average number of activations of T cells by S^{δT}-mRNA-LNP according to embodiments of the present disclosure.

FIGS. 32A and 32B show distributions of firefly luciferase at several time points after intramuscularly injecting linear mRNAs with different structures in mRNA-LNP forms individually to compare the performance of mRNAs with uncapped/capped and UTP/NlmψTP, according to embodiments of the present disclosure.

FIG. 33 shows total fluorescence at several time points after injecting linear mRNAs with different structures in mRNA-LNP forms individually to compare the performance of mRNAs with uncapped/capped and UTP/NlmψTP, according to embodiments of the present disclosure.

### DETAILED DESCRIPTION

Reference will now be made in detail to exemplary embodiments, examples of which are illustrated in the accompanying drawings. The implementations set forth in the following description of exemplary embodiments do not represent all implementations consistent with the present disclosure. Instead, they are merely examples of apparatuses and methods consistent with aspects related to the present disclosure as recited in the appended claims.

Uncapped mRNA and Structure Thereof

In embodiments of the present disclosure, the uncapped mRNA with a linear structure sequentially formed by regions A, B and C, each of the regions A, B and C including one or more of an adenine ribonucleotide, a guanine ribonucleotide, a cytosine ribonucleotide and a uracil ribonucleotide, where the region A is a 5′-untranslated region (UTR) comprising an internal ribosome entry site (IRES) for mediating internal entry of a ribosomal subunit so as to guide translation of the mRNA; the region B is a coding region for the translation so as to generate a protein; and the region C is a poly A region for mediating a translation efficiency of the mRNA and enhancing stability of the mRNA.

Different from the conventional capping, the embodiment of the present disclosure adopts the IRES with a similar function to the cap. IRES facilitates ribosome assembly and initiates translation by recruiting different trans-acting factors, making it possible for protein translation initiation to occur independently of the 5′ cap structure of the mRNA, presenting higher expression efficiency than that of the capping in the conventional method. For technical implementation, the IRES sequence is introduced into a plasmid, one end of the IRES sequence is connected with a promoter sequence, and the other end of the IRES sequence is connected with a gene of interest (GOD to construct a recombinant plasmid, which is used for amplifying the DNA template encoding desired mRNAs, or is taken, after linearization, as the DNA template for amplifying the desired mRNAs, and thus producing linearly desired mRNAs by transcription of the DNA template finally.

In related art, it is common to replace UTP with NlmψTP. By contrast, the embodiment of the present disclosure still uses UTP, rather than replacing it with NlmψTP, as materials for mRNA preparation, presenting high efficiency on preparation and being capable of reducing material cost of in vitro transcription (IVT).

The conventionally prepared mRNA includes a 3′UTR between the coding region and poly A region, and previous research demonstrated that the 3′UTR enhances stability of mRNA. By contrast, in embodiments of the present disclosure, the 3′UTR is removed from the prepared mRNA, but the prepared mRNA still presents high expression in the translation and stability as that of the conventionally prepared mRNA.

In summary, the uncapped mRNA provided in embodiments of the present disclosure with a new and simple structure could be prepared easily with lower cost, and still presents high expression in the translation. New mRNA-LNP is then developed on the basis of such an uncapped mRNA, with which relevant vaccine or medicaments may be further prepared.

### Example 1. Recombinant Plasmid Construction

1. The recombinant plasmid was based on pT7AMP and was constructed by the Inventors.
2. The GOIs as follows were synthesized by GenScript (Nanjing, China):
i. a truncated D-type envelope glycoproteins coding gene (gD^{ED}) from herpes simplex virus type 2 (HSV2);
ii. a truncated D-type envelope glycoproteins coding gene (gD^{FR}) from HSV2;
iii. a mutated gene (S^{δT}) encoding Delta strain SARS-CoV-2 spike protein;
iv. a firefly luciferase coding gene (Flue); and
v. an enhanced green fluorescent protein coding gene (eGFP).

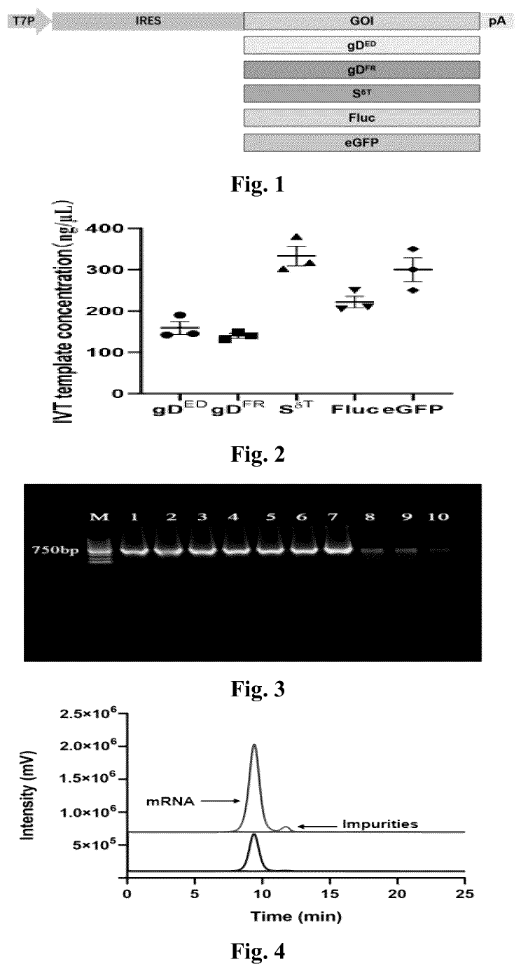

The sequence of gD^{ED} in (i) is set forth in SEQ ID NO: 1, which encodes HSV2 gD^{ED} with 316 amino acids. The sequence of gD^{FR} in (ii) is set forth in SEQ ID NO: 2, which encodes HSV2 gD^{FR} with 400 amino acids. The mutated gene S^{δT} in (iii) has a sequence set forth in SEQ ID NO: 3, encoding mutated SARS-CoV-2 spike protein from Delta strain (SARS-CoV-2-S^{δT}), including proline mutations among extracellular domain of the strain at positions 1-1202aa, RRAR mutated to GGSG at 682-685aa for S1/S2 cleavage site and a trimerization domain.
3. The sequence for poly A (106 bp) were synthesized by GenScript (Nanjing, China).
4. As shown in FIG. 1, the above synthesized sequences were individually cloned into the ampicillin-resistant pT7AMP vector by homologous recombination, with BgIII sites as restriction of enzyme endolucent sites, to obtain 5 recombinant plasmids of pT7AMP-gD^{ED}, pT7AMP-gD^{FR}, pT7AMP-S^{δT}, pT7AMP-Fluc, and pT7AMP-eGFP. All of the recombinant plasmids were verified by sequencing.
5. The verified recombinant plasmids were transformed into Escherichia coli followed by selecting a single colony to be cultured in LB medium containing ampicillin (100 μg/mL, purchased from Biosharp, cat. BS923-5g) at 37° C. and shaking at 200 rpm for 16-24 hours. After that, the recombinant plasmids were extracted with a kit (Vazyme Biotech Co., Ltd, DC201-01) and stored in aliquots at −20° C.

Results

As shown in FIG. 1, each of the recombinant plasmids, namely element sequences of the mRNA structure, includes a T7 promoter (T7P), internal ribosome entry sites (IRES), a gene of interest (GOI) and a poly A region. The poly A region contains 106 duplicated adenosine triphosphates, and the GOIs (i.e. gD^{ED}, gD^{FR}, S^{δT}, Fluc and GFP) were each inserted between the IRES and poly A sequences in the pT7AMP by homologous recombination. All constructed plasmids were verified by sequencing.

### Example 2. Preparation of Template DNA for mRNA Linear Transcription

1. The recombinant plasmids described above were used as templates, and a primer pair including T7P-F (set forth in SEQ ID NO: 4) and pA-R (set forth in SEQ ID NO: 5) synthesized by Wuhan Qingke Biotechnology Co., Ltd.) was used for PCR amplification.

2. PCR amplifications were performed with 2× Phanta® Flash Master Mix (Vazyme Biotech Co., Ltd., P510-03) to obtain mRNA linear transcription templates. The PCR amplification system and procedure are shown in Tables 1 and 2 respectively.

TABLE 1

| Component | Volume | |
|---|---|---|
| 2 × Phanta ® Flash Master Mix | 25 | μl |
| Template DNA | 1 | μl |
| T7 P F (10 μM) | 1 | μl |
| pA R (10 μM) | 1 | μl |
| ddH₂O | to 50 | μl |

TABLE 2

| Step | Temperature | Time | |
|---|---|---|---|
| 1 | 98° C. | 5 | min |
| 2 | 98° C. | 10 | sec |
| 3 | 55° C. | 30 | sec |
| 4 | 72° C. | 35 | sec |
| | 2-34 cycles for steps 2-4 | | |
| 5 | 72° C. | 5 | min |

3. The PCR product was taken for concentration detection by using a Qubit 4.0 Fluorometer (Thermo) with a Qubit® DNA BR Assay. The PCR product quality was also assayed by electrophoresis on a 1% agarose gel at 120 V for 30 minutes followed by visualization with a gel imaging system.

### Example 3. In Vitro Transcription (IVT) of the Template DNA

1. IVT system for linear mRNA was optimized by design of experiment (DOE). The optimized reaction system (200 μL) was as: ATP for 5 mM (CAT: 04980824, Roche, Mannheim), CTP for 5 mM (CAT: 04980859, Roche, Mannheim), GTP for 5 mM (CAT: 04980859, Roche, Mannheim), UTP for 5 mM (CAT: 049879818, Roche, Mannheim), recombinant T7 RNA polymerase for 1200 U (CAT: 08140669, Roche, Mannheim), DNA template for 4-7 inorganic pyrophosphatase for 3 U (CAT: 08140677, Roche, Mannheim), RNase inhibitor for 40 U (CAT: GMP-E125-HC, Novoprotein Biotechnology Co., Ltd.), 10× Transcription Buffer for 20 μl (recipe: 400 mM Tris-HCl with pH=7.9, 240 mM MgCl 2, 20 mM spermidine, 100 mM DTT and DEPC water). To synthesize NlmψTP-modified mRNA, UTP (short for U) was replaced with NlmψTP (CAT: GPNPU-20210802, Wuhan Tangzhi Biotechnology Co., Ltd., Wuhan, China).
2. After reacting for 3 hours at 37° C., DNase I (Novoprotein Biotechnology Co., Ltd.) was added into the reaction system to degrade the DNA in the system, to obtain primary mRNA products from IVT. The primary mRNA products were aliquoted and stored at −80° C.

Results

The IVT DNA templates were obtained by PCR amplification by using primer pairs (T7-PF and pA-R) and the above five recombinant plasmids individually as templates. The electrophoresis results showed that the five IVT template fragments presented all single bands with strong brightness, which was consistent with the expected fragment sizes (i.e. 1834 bp, 2075 bp, 1670 bp, 2528 bp, and 4657 bp for the IVT templates of 5 GOIs of gD^{ED}, gD^{FR}, S^{δT}, Fluc and eGFP, respectively), without other obvious bands having undesired sizes. As shown in FIG. 2, the concentration of IVT templates was 240±100 ng/μL, as determined by UV spectrophotometry or the Qubit method. Repeated trials showed that the IVT templates with relatively high concentration and purity could be rapidly obtained by PCR amplification.

### Example 4. Purification with Chromatographic Column

The primary mRNA product was purified with an AKTA Avant chromatography system (GE, Sweden) and a CIMmultus™ Oligo dT 18 chromatography column (BIA Separations, Slovenia). Specifically, the primary mRNA product was mixed with OA (Oligo dT Adjust) buffer (recipe: 50 mM phosphate buffer, 2 mM EDTA and 1 M NaCl) at a ratio of 4:1 to adjust the ionic concentration of the sample solution. The mixed sample was injected into the AKTA sample loop, by which the mRNA product was adsorbed. After that, the column was washed with OW (Oligo dT Wash) buffer (recipe: 50 mM phosphate buffer and 2 mM EDTA). The adsorbed mRNA was eluted from the column with water to obtain the purified mRNA stock solution, of which the concentration and purity were detected by SEC-HPLC (SHIMADZU, Japan) with an analytical TSK G6000 PWXL column (CAT: 0008024, TOSOH, Japan). The mobile phase was 0.02 M phosphate buffer at a flow rate of 1 mL/min.

Results

The concentration and purification of the primary mRNA obtained by IVT were determined by electrophoresis (for concentration and purification determination) and the Qubit assay (for concentration determination only). As shown in FIG. 3, the mRNAs synthesized by 7 duplicate IVT experiments individually were loaded in lanes 1-7 for 1 μl, and all of them are Fluc mRNAs; “M” referred to a DNA marker within a range of 5000 bp (Vazyme, China) for 5 μl; and lanes 8, 9 and 10 were 900, 500, and 250 ng RNA references, respectively. The concentrations of mRNA synthesized by 7 duplicate IVT experiments were all within 4.3±0.5 mg/mL, indicating that the IVT system optimized by DOE could produce primary mRNAs in a high and robust manner.

The primary mRNA synthesized by IVT was then purified by a CIMmultus™ Oligo dT 18 chromatographic column, and a small amount of the purified mRNA was tested with HPLC for detect its purity and concentration. The SEC-HPLC test results showed that the number and area of impurities peaks in the purified mRNA samples were reduced, and the mRNA single peak area accounted for 99% of the total peak area (FIG. 4).

### Example 5. Preparation of Lipid Nanoparticles (mRNA-LNPs)

1. Four components, including SM 102 (06040008800, Xiamen Sinopeg), DSPC (1,2-distearoyl-sn-glycero-3-phosphocholine, purchased from AVT, S01005), cholesterol (AVT, 001001) and DMG-PEG2000 (purchased from AVT, 002005), were dissolved in ethanol at a molar ratio of 50:10:38:2, to obtain an ethanol phase.
2. The ethanol phase was then mixed with sodium acetate buffer (25 mM, pH=5.0) with mRNA at 1:4 (ethanol phase:water phase) in a microfluidic mixer (Precision Nanosystems, Canada or NanoPro System, self-developed). The ionizable N:P ratio in the formulation was 4:1.
3. The encapsulated mRNA-LNPs were concentrated by changing the buffer (20 mM Tris solution containing 8% sucrose, pH=7.4) with a centrifugal filter (Millipore, 30 kD) or a filtration product (Sartorius, 30 kD). The mRNA-LNP concentrates were sterilized through a 0.22 μm filter and stored in aliquots at −70° C.
4. Detection of mRNA encapsulation efficiency

The mRNA concentrations were measured on a Qubit 4.0 Fluorometer (Thermo) with a Qubit® RNA BR Assay kit (Thermo). According to the instructions, an unencapsulated mRNA content (CO and an mRNA content (Ct) of the same sample after demulsification by treating with 0.1% (v/v) Triton X-100 (Sigma) were measured to calculate the mRNA encapsulation efficiency in percentage (encapsulation percentage, EN %). The EN % was calculated as (1−Cf/Ct)×100%.

Results

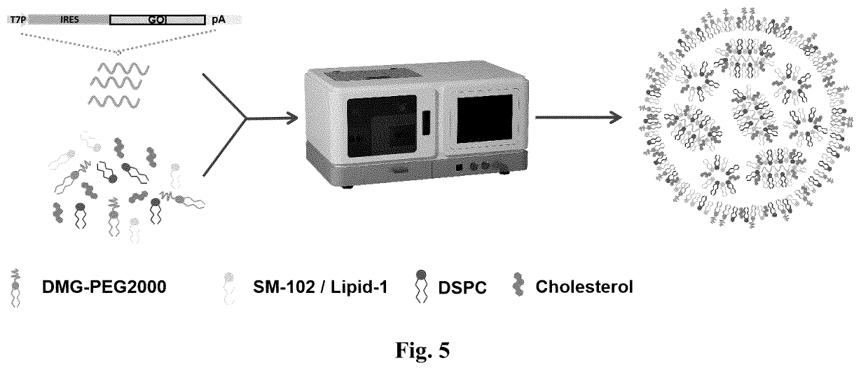

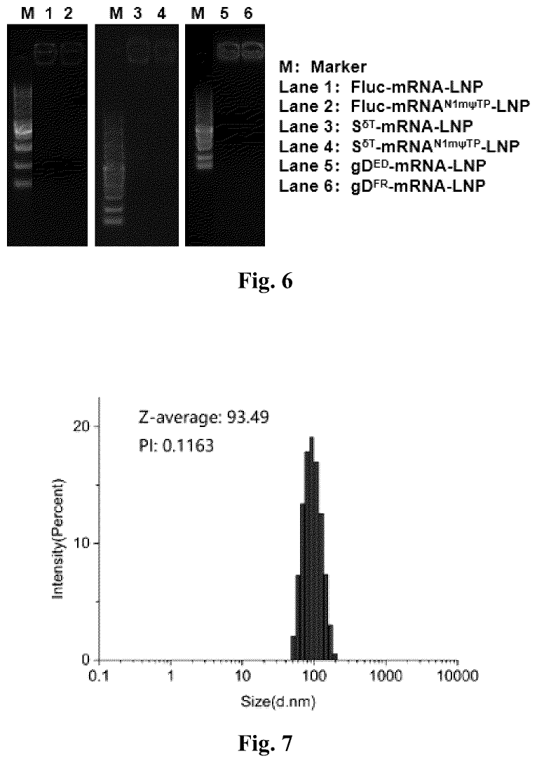

Six uncapped mRNAs, including gD^{ED}-mRNA, gD^{FR}-mRNA, S protein mRNA from the Delta strain (S^{δT}-mRNA), S^{δT}-mRNA^{N1mψTP} in which UTP was replaced by NlmψTP, Fluc-mRNA, and Fluc-mRNA^{N1mψTP} in which UTP was replaced by NlmψTP, were encapsulated by LNPs. As shown in FIG. 5, the uncapped mRNAs were mixed with the lipids in a microfluidic mixer thereby generating the mRNA-LNPs, respectively. The mRNA-LNPs were analyzed by 1% agarose gel electrophoresis at 120 V for at least 30 minutes, and it could be seen from FIG. 6 that all encapsulated mRNAs could not be removed and remained in the loading wells, and mRNA bands of expected sizes were not showed in corresponding lanes. The results of mRNA concentration measurements showed that the encapsulation efficiency of the six uncapped mRNAs was greater than 90%.

Table 3 shows the unencapsulated mRNA content (Cf), total mRNA content (Ct) and encapsulation efficiency values after encapsulation of six of the uncapped mRNAs. After removal of the nonaqueous solvent in the encapsulation system, each mRNA-LNP had a particle size (Z average) between 70 and 100 nm and a polymer dispensability index (PDI) less than 0.2 (FIG. 7).

TABLE 3

| | C_{f} | Cₜ | EN | Z-average | |
|---|---|---|---|---|---|
| | (ng/μl) | (ng/μl) | (%) | (nm) | PDI |
| mRNA-Fluc^{UTP} | 7.18 | 217 | 96.3 | 79.9 | 0.09 |
| Fluc-mRNA^{N1mψTP}-LNP | 5.44 | 258 | 97.9 | 74.5 | 0.12 |
| S^{δT}-mRNA-LNP | 5.54 | 290 | 98.1 | 80.9 | 0.07 |
| S^{δT}-mRNA^{N1mψTP}-LNP | 5.40 | 300 | 98.2 | 84.1 | 0.06 |
| gD^{ED}-mRNA-LNP | 11.0 | 320 | 96.5 | 85.6 | 0.08 |
| gD^{FR}-mRNA-LNP | 12.0 | 265 | 95.4 | 86.7 | 0.07 |

### Example 6. In Vitro Transfection of mRNA-LNP

1. Materials for in vitro transfection were as follows: BHK (Baby Hamster Kidney, purchased from ATCC); fetal bovine serum (FBS, purchased from Zhejiang Tianhang Biotechnology Ltd., 110-11-86-11); high-glucose DMEM (purchased from Procell, PM150210); and 6-well plates (purchased from BIOFIL, TCP10006).
2. BHK cells were seeded in 6-well plates at 2×10⁵ cells/well. After 24 hours, when the cell confluence reached 6070%, the medium in the wells was replaced with 2 mL of fresh high-glucose DMEM containing 5% FBS. The mRNA-LNPs at 3˜4 μg mRNA per well were slowly added along the wall of the well and shaken evenly. The cells were cultured at 37° C. and 5% CO₂ for 4 hours, followed by observation of the cells transfected with the mRNA-LNPs.

Results

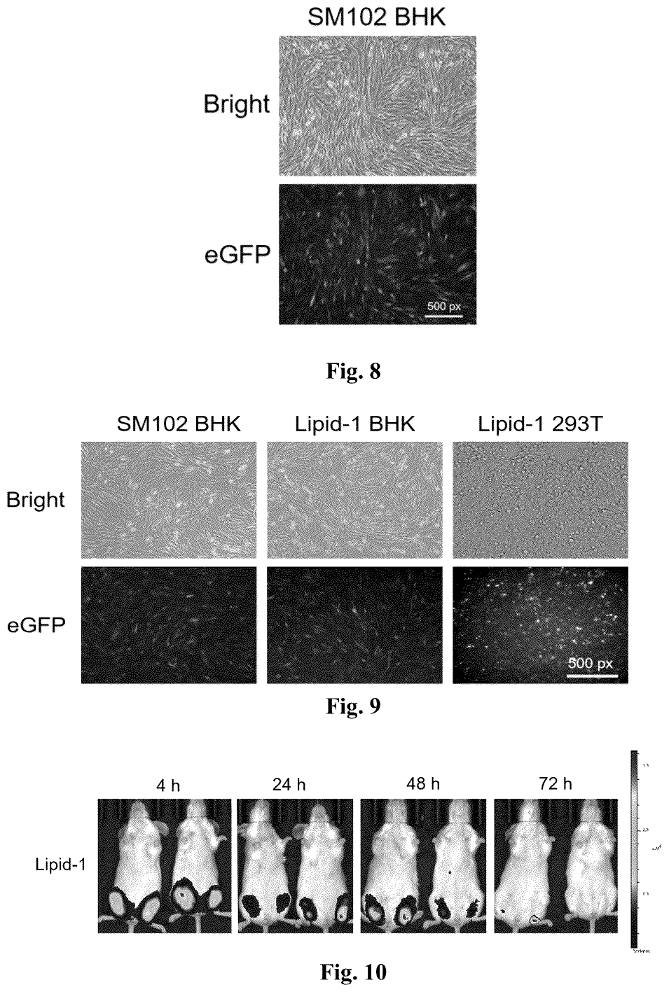

The uncapped eGFP-mRNA-LNPs could transfect a variety of cells, and the transfection method is simple and robust. Taking BHK cells as an example, after 4 hours of transfection with the eGFP-mRNA-LNPs (without requirements for removing eGFP-mRNA-LNPs and changing the medium), it could be observed that the green fluorescent protein (eGFP) was expressed in the cells, and the cells presented normal morphology with clear boundary (FIG. 8). The results showed that the uncapped eGFP-mRNA encapsulated by LNPs can quickly enter the cytoplasm through endocytosis and efficiently translate the target protein, and the eGFP-mRNA-LNP did not show obvious cytotoxicity. The uncapped eGFP-mRNA-LNP can also be transfected into cells such as HEK 293T. The eGFP-mRNA-LNPs encapsulated with either SM102 or self-developed cationic lipids (Lipid-1) could easily and efficiently transfect cells to produce eGFP (FIG. 9).

### Example 7. Animal Experiments In Vivo with mRNA-LNPs

1. Animal Sources

All animal experiments were strictly carried out in accordance with the requirements of the Hubei Provincial Committee for the Management and Use of Laboratory Animals.

Female C57BL/6 mice six to eight weeks old (Certificate No. 42000600043578), 5-week-old female Syrian hamsters (Certificate No. 42000400012878), and 5-week-old female Japanese white rabbits were all purchased from the Hubei Provincial Center for Disease Control and Prevention.

Animals had free access to water and food in a controlled environment with 12 hours in light, 12 hours in dark.

2. Animal Model Construction

2.1 Fluc-LNP Animal Model

Each of linear mRNAs encapsulated by LNP, namely Fluc-mRNA^{UTP}-LNP and Fluc-mRNA^{N1mψTP}-LNP, was injected intramuscularly in the thighs of C57BL/6 mice. After 4 hours, 100 μL of D-fluorescein potassium salt (Beyotime, 15 mg/mL) was intraperitoneally injected, and images were taken with an IVIS imaging system (Perkin Elmer) in 10 minutes.

2.2 Immunization with S^{δT}-mRNA-LNP

At days 0 and 14, S^{δT}-mRNA encapsulated by LNP (i.e. S^{δT}-mRNA-LNP) was injected into the thigh muscle of C57BL/6 mice.

At days 0 and 21, S^{δT}-mRNA encapsulated by LNP (i.e. S^{δT}-mRNA-LNP) was injected into the thigh muscle of hamsters.

Orbital blood and heart blood samples were collected from mice and hamsters, respectively, before and after the immunization, and the serum samples were separated for the detection of IgG antibody titers and/or neutralizing antibody titers.

2.3. Immunization with gD-mRNA-LNP

At days 0, 14 and 49, each of linear gD-mRNAs encapsulated by LNP (i.e. gD^{ED}-mRNA-LNP and gD^{FR}-mRNA-LNP) was injected into the thigh muscle of C57BL/6 mice. Orbital blood samples were collected from mice, before and after the immunization, and the serum samples were separated for the detection of neutralizing antibody titers.

At days 0 and 14, different doses of gD-mRNAs encapsulated by LNP (i.e. gD′-mRNA-LNP and gD′-mRNA-LNP) were injected into the Japanese white rabbits at the posterior neck intradermally and at the hind leg intramuscularly, respectively. Before and after the immunization, venous blood samples were collected from rabbit ear and serum were separated for the detection of neutralizing antibody titers.

3. ELISA Detection of Antibodies

3.1 Detection of IgG Expression Specifically in Responsive to Spike (S) Protein Trimerization from SARS-CoV-2

a. Reagents

SARS-CoV-2 (B.1.617.2) spike (S) protein was purchased from Nanjing Vazyme Biotechnology Co., Ltd. (GC221-01) with an initial concentration of 2.6 mg/ml. Tween-20 was purchased from Sinopharm Chemical Reagent Co., Ltd. (Cat. No. 30189328). Bovine serum albumin (BSA) was purchased from BioFroxx (Cat. No. 4240GR025); 96-well microtiter plate was purchased from Thermo (Cat. No. 446469); and sulfuric acid was purchased from Sinopharm Chemical Reagent Co., Ltd. (Cat. No. 10021628).

b. Experimental Procedure

The microtiter plate was coated with SARS-CoV-2 (B.1.617.2) S protein at 100 ng/well and placed overnight at 4° C. The next day, the plate was washed with a washing solution (PBST containing 0.05% Tween-20) and blocked with PBST containing 1% BSA for 2 hours.

The serum samples of C57BL/6 mice were serially diluted 2-fold starting from 1:1000, while the serum samples of hamster were diluted to 1:2×10⁴ and then serially diluted 2 fold. The diluted serum samples at several concentrations were sequentially added to the blocked plates for incubation at 37° C. for 60 minutes. After washing the plate 3 times with the washing solution, corresponding diluted solution of horseradish peroxidase (HRP)-conjugated antibody (goat anti-mouse IgG 1:2000, Proteintech, SA00001-1; or rabbit anti-hamster IgG 1:5000, Jackson ImmunoResearch, 307-035-003) was added followed by incubation at 37° C. for 60 minutes. After fully washing the plate with the washing solution, TMB solution (Solarbio, PR1200) was added for incubation at 37° C. for 5 minutes. Next, coloration of each well was stopped with 2 M sulfuric acid. The absorbance at a wavelength of 450 nm of each well was read with a microplate reader (Thermo).

4. Detection of Neutralization Antibodies

Neutralization of serum antibodies was assessed by a constant virus amount. Serum samples were serially diluted 2-fold starting from 1:8. Each of the diluted serum samples was thoroughly mixed with the oHSV2-eGFP (in house) virus solution containing 100 CCID₅₀ to obtain a serum-virus mixture that was incubated at 37° C. for 1 hour in the presence of penicillin (100 U/mL) and streptomycin (0.1 mg/mL) supplemented according to volume of the mixture. Then, each serum-virus mixture was inoculated into the corresponding well of a 96-well microtiter plate cultured with a monolayer of Vero cells and incubated at 37° C. in 5% CO 2 for 48 hours. Fluorescence expression in the wells was observed using the High Content Analysis System (Perkin Elmer, PE) respectively. Antibody neutralization titers were assessed based on the GFP expressions.

The neutralizing activity of serum antibodies was assessed by constant serum dilutions. The oHSV2-eGFP virus was serially diluted 2 fold from 1:2 to obtain suspensions containing different amounts of virus (1500-100000 CCID₅₀). Each virus suspension was thoroughly mixed with serum to obtain a virus-serum mixture that was incubated at 37° C. for 1 hour in the presence of penicillin (100 U/mL) and streptomycin (0.1 mg/mL) supplemented according to volume of the mixture. Then, each virus-serum mixture was inoculated into the corresponding well of a 96-well microtiter plate cultured with a monolayer of Vero cells and incubated at 37° C. in 5% CO₂ for 48 hours. Fluorescence expression and cytopathic effect in wells detected by the High Content Analysis System were used to assess the neutralizing activity of serum antibodies.

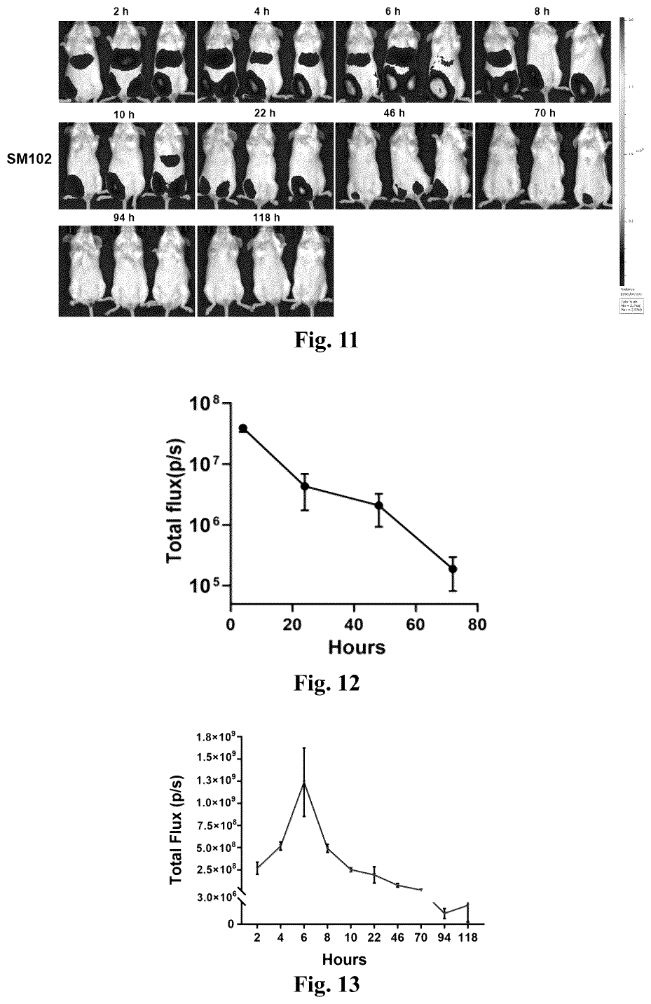

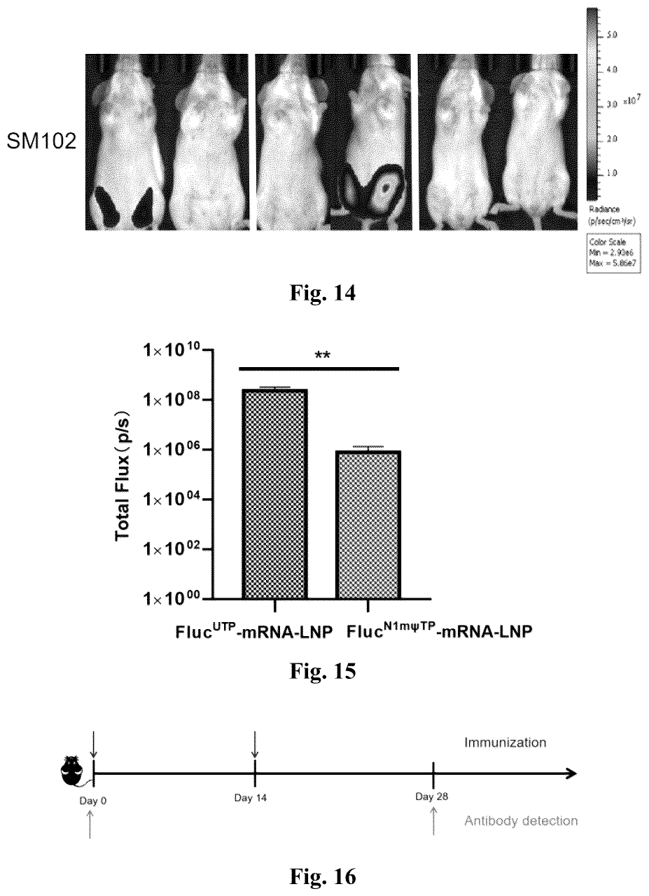

Results
1. In vivo transfections of uncapped Fluc-mRNA-LNPs encapsulated with SM102 and Lipid-1 (developed independently by the Inventors) respectively presented durable and high-yield expression. As shown in FIGS. 10 and 11, the Fluc-mRNA-LNPs encapsulated with SM102 and Lipid-1 individually were administered intramuscularly to the mice (n=5 for Lipid-1; n=3 for SM102), and the fluorescence intensity by exciting Fluc in the mice was observed at different time points. The fluorescence intensity near the injection site first increased and then decreased with time, and continued in vivo for at least 72 hours for Lipid-1 and 94 hours for SM102 (FIGS. 12 and 13). These results indicated that the encapsulated Fluc-mRNA with uncapped structure can be expressed efficiently in vivo in mice.
2. As shown in FIGS. 14 and 15, four hours after intramuscular injection to C57BL/6 mice, the mean fluorescence intensity (p/s) of the Fluc-mRNA^{N1mψTP}-LNP group was 8.04×10⁵±1.43×10⁶ (mean±SEM, n=8), while that of the Fluc-mRNA^{UTP}-LNP group was 2.29×10⁸±1.04×10⁸ (mean±SEM, n=4), indicating that the uncapped mRNA-LNP with UTP realized a higher expression.
3. Defining the day of the first immunization as day 0, C57BL/6 mice were immunized on days 0 and 14 (FIG. 16). Three groups, namely a UTP group (30 μg/100 μL/animal, n=3); an NlmψTP group (30 μg/100 μL/animal, n=3); and a naïve group (without immunization, n=3), were set. On the 28^{th} day, blood samples were collected to separate the serum for detecting the serum IgG antibody titer.

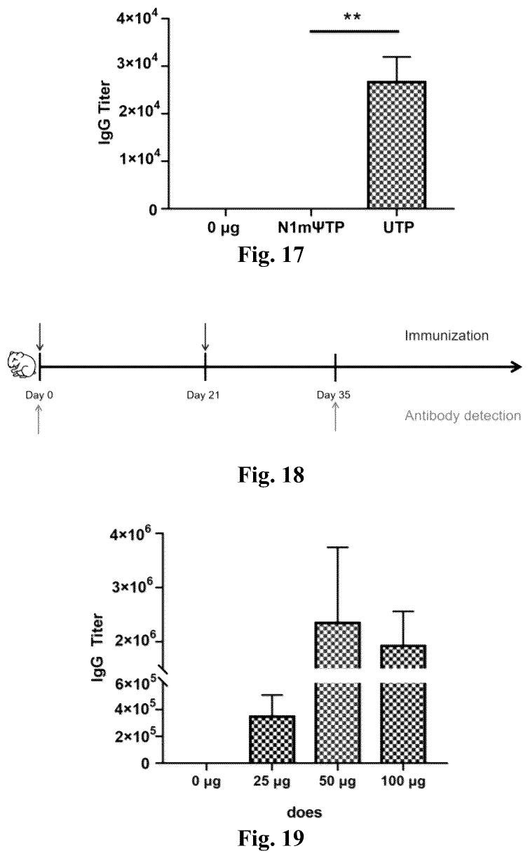

The results showed that the average titer of the UTP group reached 1:26666, whereas the titers of the NlmψTP group and the naïve group were 0 (FIG. 17). Statistical analysis showed that the titer of the UTP group was significantly different from that of the naïve group and the NlmψTPU group (P=0.0048; t-test).
4. Defining the day for the first dose as day 0, and Syrian hamsters were immunized with an intramuscular injection of S^{δT}-mRNA-LNP on days 0 and 21 (FIG. 18). Four groups, i.e., a 25 μg-UTP group (25 μg/100 μL/animal, n=3); a 50 μg-UTP group (50 μg/100 μL/animal, n=3); a 100 μg-UTP group (100 μg/100 μL/animal, n=3); and a naïve group (without immunization, n=3). On the 35^{th} day, blood samples were collected to separate serum for detecting serum IgG antibody titer.

As shown in FIG. 19, the mean titers of IgG antibodies in the three treatment groups with dose of 25 μg, 50 μg, and 100 μg were (3.47±1.62)×10⁶, (2.35±1.40)×10⁷ and (1.92±0.64)×10⁷, respectively.

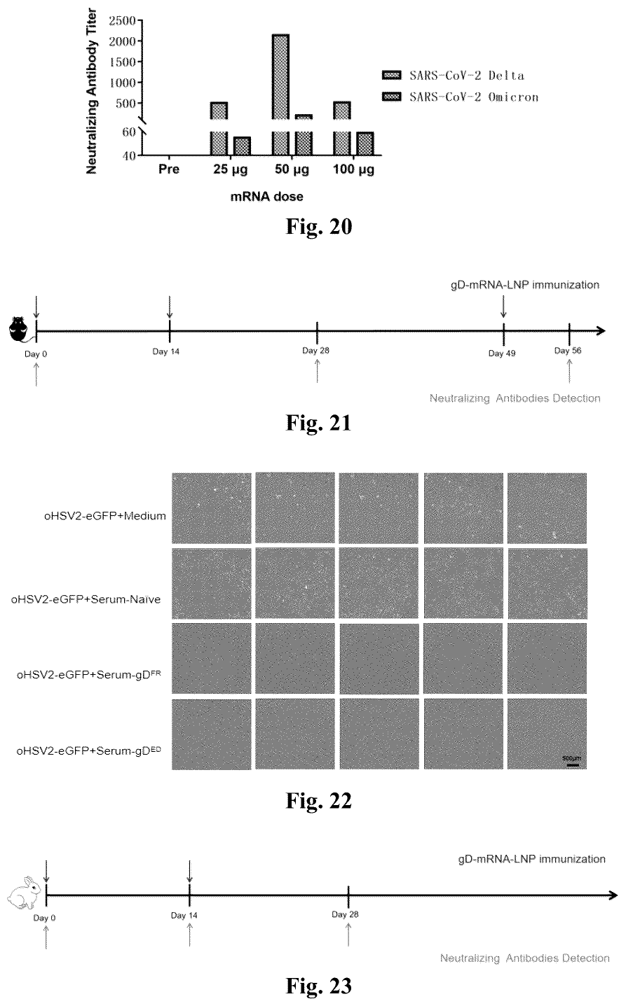

The antibodies raised in Syrian hamsters with injections of S^{δT}-mRNA-LNP in vivo can effectively neutralize both Delta and Omicron strains of SARS-CoV-2, as shown in FIG. 20.
5. Animals immunized with gD^{ED}-mRNA-LNP and gD^{FR}-mRNA-LNP produced potent neutralizing antibodies

Defining the day for the first immunization as day 0, on days 0, 14 and 49, mice were injected intramuscularly with gD^{ED}-mRNA-LNP (30 μg/100 μL/mouse, n=5) or gD^{FR}-mRNA-LNP (30 μg/100 μL/mouse, n=5) (FIG. 21).

On days 0, 28, and 52, blood samples were collected to separate sera for the detection of neutralizing antibodies. Specifically, 1 μl of serum of each samples was mixed well with oHSV2-GFP virus containing 6250 CCID₅₀ and incubated for 1 hour, followed by inoculating into wells containing a monolayer of Vero cells. The neutralizing titer of antibodies was assessed by expressions of GFP.

As shown in FIG. 22, all cells treated with the serum-virus mixture in the unimmunized group produced GFP, and typical viral plaques resulting from virus replication could be seen. In contrast, GFP and viral plaques were not observed in cells treated with the serum-virus mixture (containing 6250 CCID₅₀ virus) in the gD^{ED}-mRNA-LNP and gD^{FR}-mRNA-LNP groups. The results indicated that C57BL/6 mice immunized with the two HSV2 gD antigens expressed in vivo based on the uncapped mRNA with unmodified uridine could induce neutralizing antibodies.
6. In another experiment, by defining the day for the first immunization as day 0, as shown in FIG. 23, on days 0 and 14, Japanese white rabbits were intradermally injected with 200 μL (20 μg) of mRNA-LNP-gD^{ED} into the hind neck, and the other two Japanese white rabbits were intramuscularly injected with 250 μl (25 μg) of mRNA-LNP-gD^{ED} and 500 μl (50 μg) of mRNA-LNP-gD^{ED} in the hind legs, respectively. On days 0, 14, and 28, blood samples were collected from the ear artery to separate the serum to detect neutralizing antibodies. Live virus neutralization titrations were performed to assess the neutralizing potency of antibodies produced by rabbits immunized with mRNA-LNP-gD^{ED}.

Results

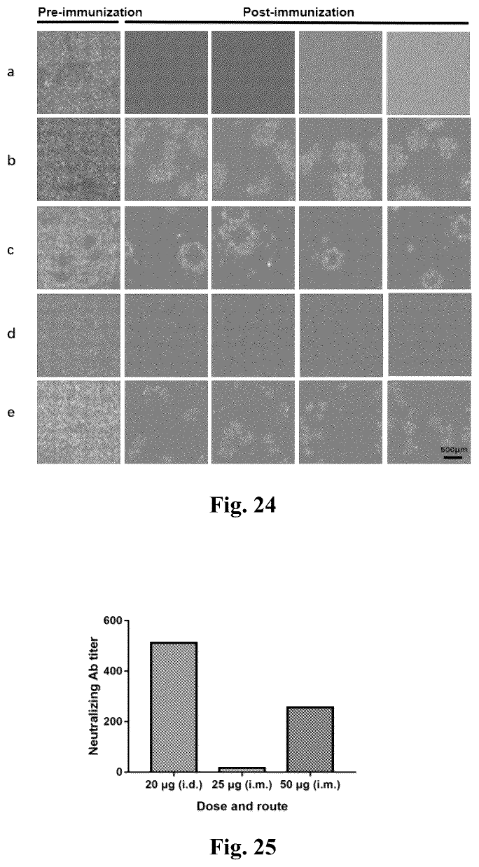

As shown in FIG. 24, by comparing the serum samples pre- and post-immunization, the serum samples post-immunization had significant neutralization activity to the virus. In rabbits injected intradermally, 1 μL of serum completely neutralized 40960 CCID₅₀ viruses containing oHSV2-GFP, without GFP expression or plaque formation, and the serum can completely neutralized 100 CCID₅₀ viruses after diluted 128-fold. The neutralization effects of the two rabbits injected intramuscularly were weaker than that of the intradermally injected rabbit. For the rabbit injected intramuscularly with 50 μg, serum did not completely neutralize the viruses and plaques appeared when diluted 64-fold; and in the 25 μg intramuscularly injected rabbit, when the serum was diluted 16-fold, the viruses could not be completely neutralized. The neutralization titers differential on the immunized dose and route are shown in FIG. 25. To exclude the effect of complement on the experiment, complement inactivation was performed in Examples of the present disclosure. The experimental results showed that the effect achieved in the complement inactivation group was the same as that in the noninactivation group, indicating that the complement had no effect on the neutralization. It was concluded that rabbit serum samples after immunization (via intradermal or intramuscular injection) of gD^{ED}-mRNA-LNP could neutralize the oHSV2-eGFP virus.

### Example 8. Cellular Immunity Activated by Uncapped Linear mRNA-LNP

8.1 Materials

A mouse IFN-γ ELISpot PLUS kit was purchased from Mabtech (Cat. No.: 3321-4APW-2); PMA+ionomycin was purchased from Shenzhen Dakewe Bioengineering Co., Ltd. (Cat. No. 2030421); SARS-CoV-2 (B.1.617.2) S Protein was purchased from Nanjing Vazyme Biotech Co., Ltd. (Cat No GC221-01); and OH2 virus derived from oHSV2 hGM-CSF (47) from the HSV2 HG52 strain was prepared by the Inventors.

8.2 On days 0, 14 and 21, gD^{ED}-mRNA-LNP, gD^{FR}-mRNA-LNP and S^{δT}-mRNA-LNP were injected intramuscularly into the thigh of C57BL/6 mice, respectively. At the given time, the spleens of those mice were removed and splenocytes were separated to detect specific T cells.

8.3 T Cells Detection

The detection of gD-specific or S^{δT}-specific T cells were performed as follows: splenocytes at 3-5×10 5 splenocytes/well and 10⁶ OH2 virus at CCID₅₀/well inactivated by UV for 30 minutes (or 4 μg/well SARS-CoV-2 S Protein) were mixed and incubated at 37° C. After 48 hours followed by washes 5 times with PBS, probe antibody (R4-6A2 biotin, 1 μg/μL) was added and incubated at room temperature for 2 hours.

After washing with PBS 5 times, alkaline phosphatase-labeled streptavidin (Streptavidin-ALP, 1:1000) was added and incubated for 1 hour at room temperature. Repeat this step once. After washing 5 times with PBS, chromogenic solution (BCIP/NBT-plus) was added and incubated at room temperature for 10 minutes, followed by stopping color development by deionized water. Once dried, the number of spots presented in the wells on the ELISpot plate was read and analyzed using an enzyme-linked spot analyzer (AID ELISpot Reader Classic, Autoimmun Diagnostika GmbH). The data was analyzed with Student's t-test by GraphPad Prism v8.0.1 software to statistically.

8.4 Results

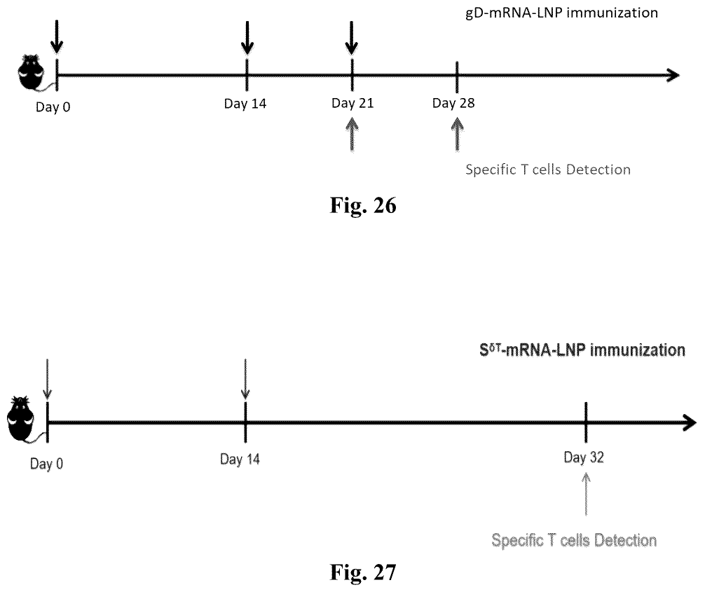

The ELISpot assay showed that animals immunized with gD^{ED}-mRNA-LNP, gD^{FR}-mRNA-LNP or S^{δT}-mRNA-LNP produced specific T cells against the specific antigens. The first immunization was recorded as day 0, and mice were immunized on designated days (FIG. 26-27). In the experiments involved with gD^{ED}-mRNA-LNP/gD^{FR}-mRNA-LNP, three groups were designated as a gD^{ED}-mRNA-LNP group (30 μg/100 μL/unit, n=8); a gD^{FR}-mRNA-LNP group (30 μg/100 μL/unit, n=8); and a naïve group (without immunization, n=8).

On day 21, the spleens of 4 mice in each group were taken for ELISpot detection, the remaining 4 mice in each group were given a second booster immunization with the same dose, and the spleens of those mice were taken on day 28 to detect specific T cells.

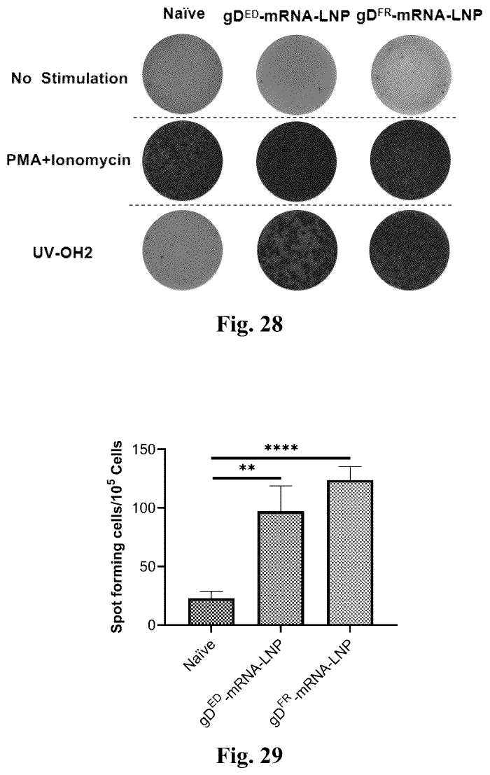

After mixing 3×10⁵ splenocytes with 10⁶ CCID₅₀ of OH2 virus inactivated by UV for 30 minutes and incubating for 48 hours, specific T cells were detected according to the number of spots formed by IFN-γ cytokines secreted by splenocytes. As shown in FIG. 28 and FIG. 29, for the data derived from spleen taken on day 28, the mean number of spots formed by IFN-γ cytokines in the two treatment groups (gD^{ED}-mRNA-LNP and gD^{FR}-mRNA-LNP groups) did have significant differences in relative to that in the naïve group.

The results indicated that the two gD antigens translated in vivo based on the uncapped mRNA with unmodified uridine could activate specific T cells in C57BL/6 mice.

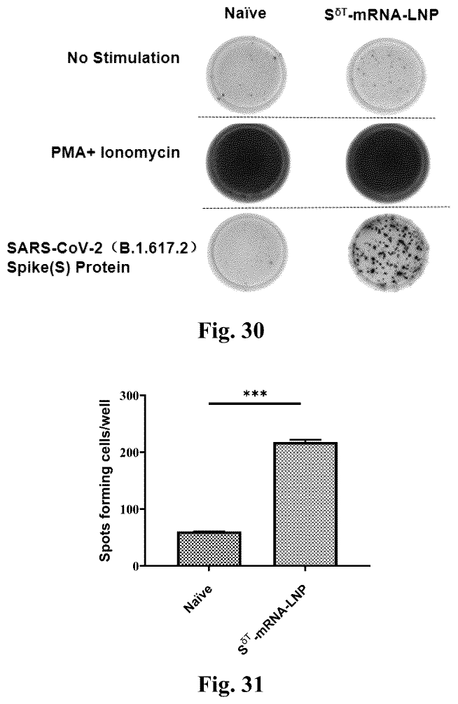

As shown in FIG. 27, the ELISpot assay was performed with a mixture of splenocytes immunized with S^{δT}-mRNA-LNP separated on day 32 and SARS-CoV-2 S protein (as stimuli). It was observed that the spike (S) protein trimerization antigens translated in vivo based on the uncapped mRNA with unmodified uridine could also activate specific T cells in C57BL/6 mice (FIG. 30-31).

### Example 9. In Vivo Transfection of Uncapped Linear mRNA and Capped mRNA

For determining delivery and expression efficiencies of mRNAs with different structures, a contrast experiment on in vivo transfection of uncapped linear mRNA and capped mRNA was performed in the Example. The construction of recombinant plasmid, preparation of mRNA-LNP, construction of animal model and etc. could refer to the corresponding methods described above.

Female BABL/c mice 7 years old were divided into three groups, i.e. a capped mRNA group, an uncapped mRNA group and a negative control. The capped mRNA group was further divided into 2 subgroups including a natural UTP capped subgroup (Cap UTP 30 μg) and a modified uridine capped subgroup (Cap N1mΨTP 30 μg), and each of the subgroups contained 6 mice. The uncapped mRNA group was further divided into 2 subgroups too including a natural UTP uncapped subgroup (Uncapped UTP 30 μg) and a modified uridine uncapped subgroup (Uncapped UTP 10 μg), and each of the subgroups contained 6 mice. The negative control included 3 mice without treatment.

Each of subgroups were intramuscularly injected with corresponding mRNA of Fluc encapsulated by LNP at corresponding doses, to observe the fluorescence intensity and duration in the mice at several points in time.

Results

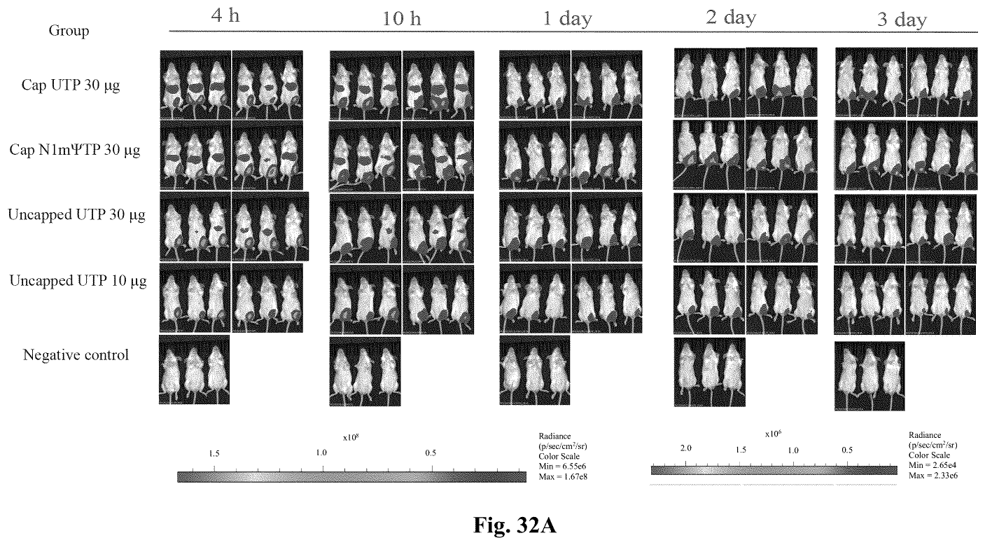

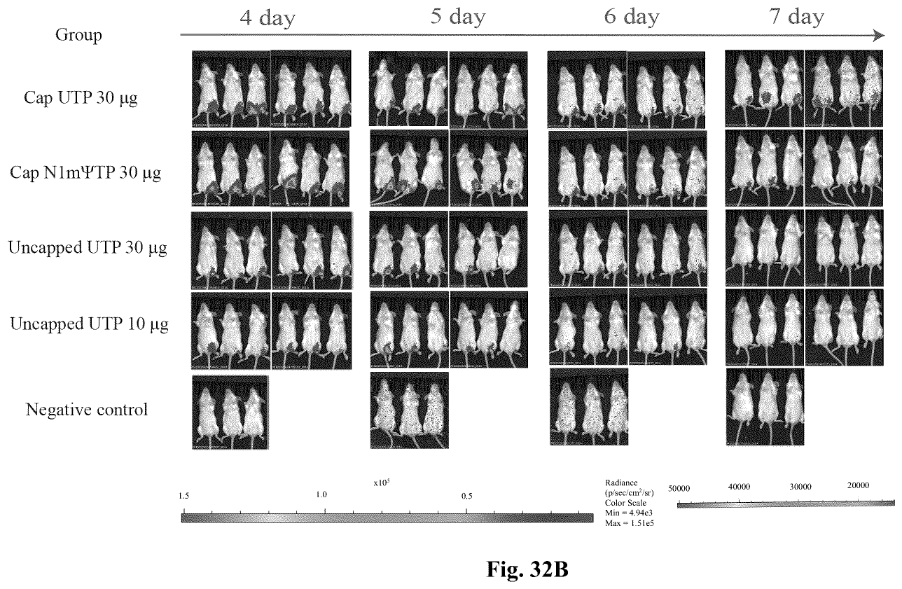

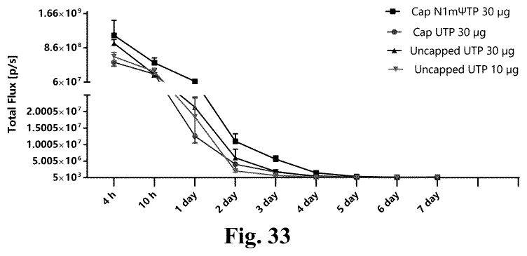

As shown in FIGS. 32A, 32B, and 33, the fluorescence intensity at both of the injection sites and liver of mice reached 10⁸ P/S (Photons per second) in 4 hours after injection, and gradually decreased with time. On day 7, the fluorescence intensity decreased to 10³ P/S, and the overall expression trend of uncapped linear mRNA and capped mRNA was consistent.

The above results showed that, in Examples of the present application, the uncapped linear mRNA with natural uridine has the same delivery in vivo and expression efficiency basically as that of the traditional capped mRNA with modified uridine, both of which can achieve efficient and lasting expression of the target gene in vivo.

It should be understood that the foregoing general description is exemplary and explanatory only and is not restrictive of the present disclosure, as claimed. It will be appreciated that the present disclosure is not limited to the exact construction that has been described above and illustrated in the accompanying drawings, and that various modifications and changes can be made without departing from the scope thereof.

What is claimed is:

1. An uncapped mRNA with a linear structure sequentially formed by regions A, B and C, each of the regions A, B and C comprising one or more of an adenine ribonucleotide, a guanine ribonucleotide, a cytosine ribonucleotide and a uracil ribonucleotide, wherein
the region A is a 5′-untranslated region (UTR) comprising an internal ribosome entry site (IRES) for mediating internal entry of a ribosomal subunit so as to guide translation of the mRNA;
the region B is a coding region for the translation so as to generate a protein; and
the region C is a poly A region for mediating a translation efficiency of the mRNA and enhancing stability of the mRNA,
wherein the region B comprises one or more of the following genes:
a truncated D-type envelope glycoprotein ectodomain coding gene (gD^{ED}) from herpes simplex virus type 2 (HSV2), as set forth in SEQ ID NO: 1;
a truncated D-type envelope glycoprotein coding gene (gD^{FR}) from HSV2, as set forth in SEQ ID NO: 2; and
a mutated gene (S^{δT}) encoding Delta strain SARS-CoV-2 spike protein as set forth in SEQ ID NO: 3.

2. A recombinant plasmid capable of being transcripted to obtain the uncapped mRNA according to claim 1, wherein the recombinant plasmid is any one of the following recombinant plasmids A, B and C,
the recombinant plasmid A comprising: a truncated D-type envelope glycoprotein ectodomain coding gene (gD^{ED}) from herpes simplex virus type 2 (HSV2), as set forth in SEQ ID NO: 1;
the recombinant plasmid B comprising: a truncated D-type envelope glycoprotein coding gene (gD^{FR}) from HSV2, as set forth in SEQ ID NO: 2; and
the recombinant plasmid C comprising a mutated gene (S^{δT}) encoding Delta strain SARS-CoV-2 spike protein as set forth in SEQ ID NO: 3.

3. An mRNA-lipid nanoparticle (LNP) comprising an uncapped mRNA according to claim 1 and an LNP.

4. The mRNA-LNP according to claim 3, wherein the LNP is one or more of octanoic acid, 8-[(2-hydroxyethyl)[6-oxo-6-(undecyloxy)hexyl]amino]-, 1-octylnonyl ester and lipid-1, wherein the lipid-1 has a structure as shown in formula I:

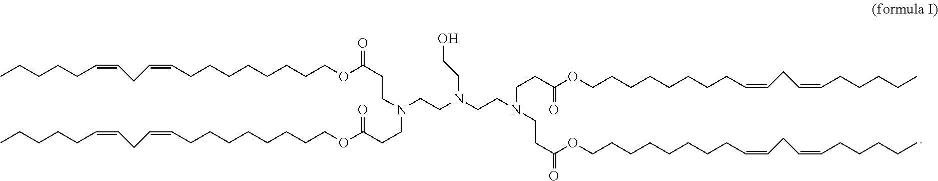 [SMILES: CCCCC/C=C\C/C=C\CCCCCCCCOC(=O)CCN(CCC(=O)OCCCCCCCC/C=C\C/C=C\CCCCC)CCN(CCO)CCN(CCC(=O)OCCCCCCCC/C=C\C/C=C\CCCCC)CCC(=O)OCCCCCCCC/C=C\C/C=C\CCCCC]

5. The mRNA-LNP according to claim 3, wherein the mRNA-LNP is prepared with the assistance of a helper molecule selected from one or more of distearoyl phosphatidylcholine, cholesterol and 1,2-dimyristoyl-rac-glycero-3-methoxypolyethylene glycol-2000.

6. The mRNA-LNP according to claim 3, wherein the helper molecule is distearoyl phosphatidylcholine, cholesterol and 1,2-dimyristoyl-rac-glycero-3-methoxypolyethylene glycol-2000.

7. The mRNA-LNP according to claim 6, wherein a molar ratio of the LNP to the helper molecule in an ethanol phase is of the cationic lipid compound, distearoyl phosphatidylcholine, cholesterol and 1,2-dimyristoyl-rac-glycero-3-methoxypolyethylene glycol-2000 at 50:10:38:2.

8. A vaccine or medicament, comprising an mRNA-LNP according to claim 3 and a pharmaceutically acceptable excipient.

9. A method for stimulating immune response in a subject, comprising administering an mRNA-LNP according to claim 3 to the subject, wherein the mRNA encapsulated by the LNP comprises an antigen coding gene selected from one or more of the follows:
a truncated D-type envelope glycoprotein ectodomain coding gene (gD^{ED}) from herpes simplex virus type 2 (HSV2), as set forth in SEQ ID NO: 1;
a truncated D-type envelope glycoprotein coding gene (gD^{FR}) from HSV2, as set forth in SEQ ID NO: 2; and
a mutated gene (S^{δT}) encoding Delta strain SARS-CoV-2 spike protein as set forth in SEQ ID NO: 3.

10. The method according to claim 9, wherein the LNP is one or more of octanoic acid, 8-[(2-hydroxyethyl)[6-oxo-6-(undecyloxy)hexyl]amino]-, 1-octylnonyl ester and lipid-1, wherein the lipid-1 has a structure as shown in formula I:

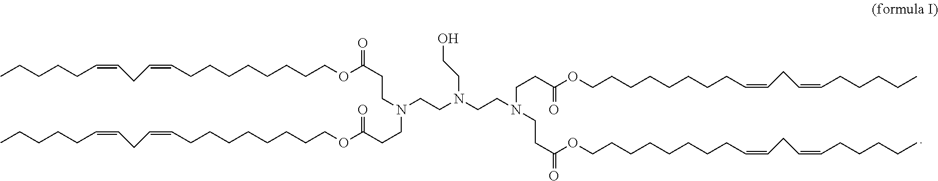 [SMILES: CCCCC/C=C\C/C=C\CCCCCCCCOC(=O)CCN(CCC(=O)OCCCCCCCC/C=C\C/C=C\CCCCC)CCN(CCO)CCN(CCC(=O)OCCCCCCCC/C=C\C/C=C\CCCCC)CCC(=O)OCCCCCCCC/C=C\C/C=C\CCCCC]

11. The method according to claim 9, wherein the subject is human or non-human.

12. The method according to claim 9, wherein the immune response comprises activation of T cells specific to the antigen.

13. The method according to claim 9, wherein the mRNA-LNP is administered intramuscularly, intracutaneously, subcutaneously, intravenously or intraperitoneally to the subject.

14. The method according to claim 9, wherein the mRNA-LNP is administered intramuscularly or intracutaneously.
